(19) United States
(12) Patent Application Publication
(10) Pub. No.: US 2012/0122903 A1
(43) Pub. Date: May 17, 2012

(54) 1-H-QUINAZOLINE-2, 4-DIONES FOR USE IN THE TREATMENT OF NEURONAL CEROID LIPOFUSCINOSIS

(76) Inventors: Hans O Kalkman, Basel (CH); Henri Mattes, Bazel (FR)

(21) Appl. No.: 13/384,280

(22) PCT Filed: Jul. 23, 2010
(86) PCT No.: PCT/EP2010/060733

§ 371 Date: Jan. 16, 2012

Related U.S. Application Data
(60) Provisional application No. 61/227,940, filed on Jul. 23, 2009.

(51) Int. Cl. A61K 31/517; C07D 403/10; A61P 25/28; C07D 239/96

(52) U.S. Cl. 514/266.23; 544/285; 514/266.3; 544/284

(57) ABSTRACT

The invention concerns the use of competitive AMPA receptor antagonists for the treatment, prevention or delay of progression of neuronal ceroid lipofuscinosis.

### FIELD OF THE INVENTION

The present invention relates to therapeutic agents for use in the treatment of neuronal ceroid lipofuscinoses.

### BACKGROUND OF THE INVENTION

The neuronal ceroid-lipofuscinoses (NCLs) are a group of inherited, neurodegenerative, lysosomal-storage disorders characterized by progressive mental and motor deterioration, seizures, and early death. Visual loss is a feature of most forms. Although neuronal ceroid lipofuscinosis was described about 100 years ago, yet currently there is no widely accepted treatment that can cure, slow down, or halt the symptoms of NCL.

Neuronal ceroid-lipofuscinoses include: infantile neuronal ceroid-lipofuscinosis (INCL. Santavuori-Haltia), late-infantile (LINCL, Jansky-Bielschowsky), Finnish late Infantile (fLINCL), Portuguese late Infantile (pLINCL), Turkish late Infantile (tLINCL), juvenile (JNCL, Batten disease, Spielmeyer-Vogt), adult (ANCL, Kuf's disease); and Northern epilepsy (NE, progressive epilepsy with mental retardation).

Children with INCL are normal at birth; symptoms usually present acutely between ages six and 24 months. Initial signs include: delayed development myoclonic jerks and/or seizures, deceleration of head growth, and specific electroencephalographic (EEG) changes. Affected infants develop retinal blindness and seizures by age two years, followed by progressive mental deterioration.

The first symptoms of LINCL typically appear between ages two and four years, usually starting with epilepsy, followed by regression of developmental milestones, dementia, ataxia, and extrapyramidal and pyramidal signs. Visual impairment typically appears at age four to six years and rapidly progresses to blindness. Life expectancy ranges from age six years to older than 40 years.

The onset of JNCL is usually between ages four and ten years. Rapidly progressing visual loss resulting in total blindness within two to four years is often the first clinical sign. Epilepsy with generalized tonic-clonic seizures, complex-partial seizures, or myoclonic seizures typically appears between ages five and 18 years. Life expectancy ranges from the late teens to the 30 s.

Initial signs and symptoms of NCL usually appear around age 30 years, with death occurring about ten years later. Affected individuals have either progressive myoclonic epilepsy or behavior abnormalities; and all have dementia, ataxia, and late-occurring pyramidal and extrapyramidal signs.

Northern epilepsy is characterized by tonic-clonic or complex-partial seizures, mental retardation, and motor dysfunction. Onset occurs between ages two and ten years.

It would be thus advantageous to provide new therapeutic agents for the treatment of neuronal ceroid lipofuscinoses

### SUMMARY OF THE INVENTION

It has been found that 1H-quinazoline-2,4-diones of formula (I)

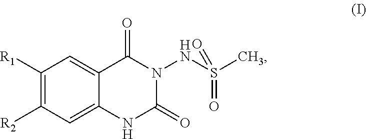

wherein
R₁ is C₁-C₆alkyl substituted by one two or three substituents selected from hydroxy, C₁-C₆alkoxy or C₅-C₆cycloalkoxy; or

### R₁ is

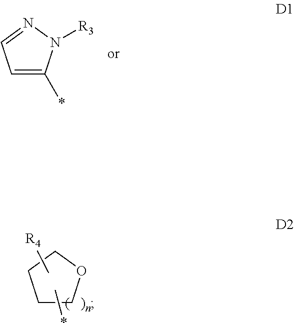

R₃ is C₁-C₆alkyl, hydroxy or C₁-C₆alkoxy-C₁-C₆alkyl;
R₄ is hydrogen or C₁-C₆alkyl;
n is 1 or 2;
R₂ is C₁-C₃alkyl or C₁-C₃fluoroalkyl;
and their pharmaceutically acceptable salts and prodrugs thereof;
may be used in the treatment, prevention or delay of progression of neuronal ceroid lipofuscinoses.

Accordingly, a first aspect of the invention concerns the use of a 1H-quinazoline-2,4-dione of formula (I) or their pharmaceutically acceptable salts or prodrugs thereof for the treatment (whether therapeutic or prophylactic), prevention or delay of progression of neuronal ceroid lipofuscinoses.

A further aspect of the invention relates to a method for the treatment, prevention or delay of progression of neuronal ceroid lipofuscinosis in a subject in need of such treatment, which comprises administering to said subject a therapeutically effective amount of a 1H quinazoline-2,4-dione of formula (I) or their pharmaceutically acceptable salts or prodrugs thereof.

A further aspect of the invention relates to a pharmaceutical composition comprising a 1H-quinazoline-2,4-dione of formula (I) or their pharmaceutically acceptable salts or prodrugs thereof for the treatment, prevention or delay of progression of neuronal ceroid lipofuscinoses.

A further aspect of the invention relates to the use of a 1H-quinazoline-2,4-dione of formula (I) or their pharmaceutically acceptable salts or prodrugs thereof for the manufacture of a medicament for the treatment, prevention or delay of progression of neuronal ceroid lipofuscinoses.

A further aspect of the invention relates to a 1H-quinazoline-2,4-dione of formula (I) or their pharmaceutically acceptable salts or prodrugs thereof for the treatment, prevention or delay of progression of neuronal ceroid lipofuscinoses

### DETAILED DESCRIPTION OF HE INVENTION

The invention relates to a compound, 1H-quinazoline-2,4-diones of formula (I),

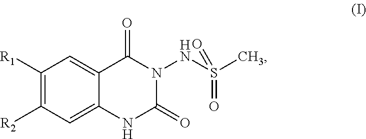

wherein
R₁ is C₁-C₆alkyl substituted by one two or three substituents selected from hydroxy, C₁-C₆alkoxy or C₅-C₆cycloalkoxy; C₅-C₆cycloalkyl substituted by one two or three substituents selected from hydroxy, C₁-C₆alkoxy or C₅-C₆cycloalkoxy; or

### R₁ is

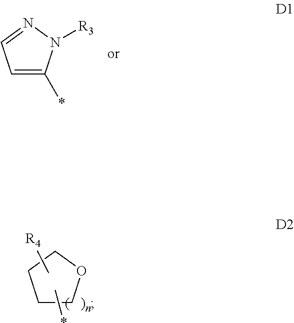

R₃ is C₁-C₆alkyl, hydroxy or C₁-C₆alkoxy-C₁-C₆alkyl;
R₄ is hydrogen or C₁-C₆alkyl;
n is 1 or 2;
R₂ is C₁-C₃alkyl or C₁-C₃fluoroalkyl;
their pharmaceutically acceptable salts, and their prodrugs thereof;
for use the treatment, prevention or delay of progression of neuronal ceroid lipofuscinosis.

The compound of formula (I) is a competitive AMPA antagonist. It is well understood that allosteric (non-competitive) antagonists provide an insurmountable blockade of AMPA receptors, potentially preventing any AMPA receptor-mediated neurotransmission at the synapse. In contrast, a high concentration of glutamate at the synapse can still activate the post-synaptic membrane in the presence of a competitive AMPA antagonist (albeit with a lower efficacy). Competitive AMPA antagonists may therefore exhibit an improved safety profile, as they will not fully block neurotransmission, but instead reduce the exaggerated glutamate signaling observed in some neurological disease.

Compounds of the formula (I) not only block AMPA-induced glutamate release from activated astrocytes but after oral dosing also suppress the symptoms associated with neuronal ceroid lipofuscinosis.

The compound of the invention of formula (I) in addition to the advantage of being a competitive AMPA antagonist receptor inhibitor, presents also the advantage of being a selective competitive AMPA antagonist. Furthermore the compound of the invention of formula (I) is capable of penetrating the blood brain barrier and may be formulated in an oral dosage form.

In the present specification, the following definitions shall apply if no specific other definition is given:

Bonds with the asterisk (*) denote point of binding to the rest of the molecule.

“C₁-C₆alkyl” represents a straight-chain or branched-chain alkyl group: for example, methyl, ethyl, n- or iso-propyl, n-, iso-, sec- or tert-butyl, n-pentyl, n-hexyl, with particular preference given to methyl, ethyl, n-propyl and iso-propyl.

“C₅-C₆cycloalkyl” represents cyclopentyl or cyclohexyl; preferably cyclopentyl.

Each alkyl/cycloalkyl-part of “alkoxy”, “cycloalkoxy”, “alkoxyalkyl” and “fluoroalkyl” shall have the same meaning as described in the above-mentioned definitions of “alkyl”/“cycloalkyl”.

“C₁-C₃fluoroalkyl” preferably represents trifluoromethyl difluoromethyl or fluoromethyl.

Compounds for use in the present invention are either obtained in the free form, as a pharmaceutically acceptable salt thereof, or as prodrug derivatives thereof.

It will be understood that any discussion of methods or references to the active ingredients includes said active ingredient in free form, in form of a pharmaceutically acceptable salt or in form of a prodrug derivative thereof. If the active ingredients have for example, at least one basic center, they can form acid addition salts if the active ingredients have for example, at least one acidic center (for example COOH) they can form salts with bases. The active ingredient or a pharmaceutically acceptable salt thereof may also be used in the form of a hydrate or may include other solvents used for crystallization.

A “pharmaceutically acceptable salt” is intended to mean a salt of a free base/free acid of a compound represented by formula (I) that is not toxic, biologically intolerable, or otherwise biologically undesirable. Preferred pharmaceutically acceptable salts are those that are pharmacologically effective and suitable for contact with the tissues of patients without undue toxicity, irritation, or allergic response. Such salts are known in the field (e.g. S. M. Berge, at al, “Pharmaceutical Salts”, J. Pharm 1977, 66:1-19; and “Handbook of Pharmaceutical Salts, Properties, Selection, and Use”, Stahl, R H., Wermuth, C. G., Eds.; Wiley-VCH and VHCA: Zurich, 2002).

In one embodiment of the invention, the 1H-quinazoline-2,4-diones of formula (I) is used in free form.

In this aspect there is provided the use of a 1H-quinazoline-2,4-dione of formula (I) for the treatment (whether therapeutic or prophylactic), prevention or delay of progression of neuronal ceroid lipofuscinoses.

In this aspect there is also provided a method for the treatment, prevention or delay of progression of neuronal ceroid lipofuscinosis in a subject in need of such treatment, which comprises administering to said subject a therapeutically effective amount of a 1H-quinazoline-2,4-dione of formula (I).

In this aspect there is also provided a pharmaceutical composition comprising a 1H-quinazoline-2,4-dione of formula (I) for the treatment, prevention or delay of progression of neuronal ceroid lipofuscinoses.

In this aspect there is also provided the use of a 1H-quinazoline-2,4-dione of formula (I) for the manufacture of a medicament for the treatment, prevention or delay of progression of neuronal ceroid lipofuscinoses.

In this aspect there is also provided a 1H-quinazoline-2,4-dione of formula (I) for the treatment, prevention or delay of progression of neuronal ceroid lipofuscinoses.

The 1H-quinazoline-2,4-diones of formula (I) and their manufacture are known from WO 2006/108591 or can be prepared analogously to said reference. WO 2006/108591 is incorporated herein by reference.

On account of asymmetrical carbon atom(s) that may be present in the 1H-quinazoline-2,4-diaries of formula (I) and their pharmaceutically acceptable salts, the compounds may exist in optically active form or in form of mixtures of optical isomers, e.g. in form of racemic mixtures or diastereomeric mixtures. All optical isomers and their mixtures, including racemic mixtures, are part of the present invention.

The term “prodrug” as used herein relates to a compound, which converts in vivo into a compound used in the present invention. A pro-drug is an active or inactive compound that is modified chemically through in viva physiological action, such as hydrolysis, metabolism and the like, into a compound of this invention following administration of the prodrug to a subject. The suitability and techniques involved in making and using pro-drugs are well known by those skilled in the art. The term “prodrug,” as used herein, represents in particular compounds which are transformed in vivo to the parent compound, for example, by hydrolysis in blood, for example as described in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987; H Bundgaard, ed, Design of Prodrugs, Elsevier, 1985; and Judkins, et al. Synthetic Communications, 26(23), 4351-4367 (1998) and The Organic Chemistry of Drug Design and Drug Action 2^{nd} Edition, R B Silverman (particularly Chapter 8, pages 497 to 557), Elsevier Academic Press, 2004

Prodrugs therefore include drugs having a functional group which has been transformed into a reversible derivative thereof. Typically, such prodrugs are transformed to the active drug by hydrolysis. As examples may be mentioned the following:

| Functional Group | Reversible derivative |
|---|---|
| Carboxylic acid | Esters, including e.g. alkyl esters |
| Alcohol | Esters, including e.g. sulfates and phos- |
| | phates as well as carboxylic acid esters |
| Amine | Amides, carbamates, imines, enamines, |
| Carbonyl (aldehyde, | Imines, oximes, acetals/ketals, enol esters, |
| ketone) | oxazolidines and thiazoxolidines |

Prodrugs also include compounds convertible to the active drug by an oxidative or reductive reaction. As examples may be mentioned:

Oxidative Activation
N-, O- and S-dealkylation
Oxidative deamination
N-oxidation
S-oxidation
Epoxidation

Reductive Activation
Azo reduction
Sulfoxide reduction
Disulfide reduction
Bio reductive alkylation
Nitro reduction.

Each of the above described reactions and/or reaction steps can be used individually or in combination in a method to prepare a AMPA-inhibitor or a prodrug thereof.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water. In one embodiment of the invention, the 1H-quinazoline-2,4-dione of formula (I) is a compound, wherein R₁ is C₁-C₆alkyl substituted by one, two or three substituents selected from hydroxy, C₁-C₆alkoxy or C₅-C₆cycloalkoxy; and R₂ is C₁-C₃alkyl or C₁-C₃fluoroalkyl.

In one embodiment of the invention, the 1H-quinazoline-2,4-dione of formula (I), its pharmaceutically acceptable salts and prodrugs thereof, is a compound, wherein R₁ is

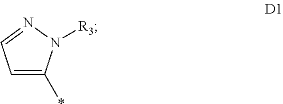

R₃ is C₁-C₆alkyl, hydroxy or C₁-C₆alkoxy-C₁-C₆alkyl; and R₂ is C₁-C₃alkyl or C₁-C₃fluoroalkyl.

In one embodiment of the invention, the 1H-quinazoline-2,4-dione of formula (I), its pharmaceutically acceptable salts and prodrugs thereof, is a compound, wherein R₁ is

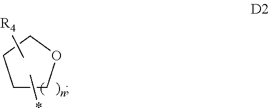

R₄ is hydrogen or C₁-C₆alkyl; n is 1 or 2; and R₂ is C₁-C₃alkyl or C₁-C₃fluoroalkyl.

In one embodiment of the invention, the 1H-quinazoline-2,4-dione of formula (I) is a compound selected from the group consisting of
A-1: N-[6-(1-Hydroxy-ethyl)-2,4-dioxo-7-trifluoromethyl-1,4-dihydro-2H-quinazolin-3-yl]-methanesulfonamide
A-2: N-[6-(1-Methoxy-ethyl)-2,4-dioxo-7-trifluoromethyl-1,4-dihydro-2H-quinazolin-3-yl]-methanesulfonamide;
A-3: N-[6-(1-Hydroxy-propyl)-2,4-dioxo-7-trifluoromethyl-1,4-dihydro-2H-quinazolin-3-yl]-methanesulfonamide;
A-4: N-[6-(1-Isopropoxy-ethyl)-2,4-dioxo-7-trifluoromethyl-1,4-dihydro-2H-quinazolin-3-yl]-methanesulfonamide;
A-5: N-[6-(1-Ethoxy-ethyl)-2,4-dioxo-7-trifluoromethyl-1,4-dihydro-2H-quinazolin-3-yl]-methanesulfonamide;
A-6: N-[2,4-Dioxo-6-(1-propoxy-propyl)-7-trifluoromethyl-1,4-dihydro-2H-quinazolin-3-yl]-methanesulfonamide;
A-7: N-[6-(1-isopropoxy-propyl)-2,4-dioxo-7-trifluoromethyl-1,4-dihydro-2H-quinazolin-3-yl]-methanesulfonamide;
A-8: N-[7-Difluoromethyl-6-(1-ethoxy-ethyl)-2,4-dioxo-1,4-dihydro-2H-quinazolin-3-yl]-methanesulfonamide;
A-9: N-[2,4-Dioxo-6-(1-propoxy-ethyl)-7-trifluoromethyl-1,4-dihydro-2H-quinazolin-3-yl]-methanesulfonamide;
A-10: N-[6-(1-Butoxy-ethyl)-2,4-dioxo-7-trifluoromethyl-1,4-dihydro-2H-quinazolin-3-yl]-methanesulfonamide;
A-11: N-[6-(1-Isobutoxy-ethyl)-2,4-dioxo-7-trifluoroethyl-1,4-dihydro-2H-quinazolin-3-yl]-methanesulfonamide;
A-12: N-[6-(1-methoxy-butyl)-2,4-dioxo-7-trifluoromethyl-1,4-dihydro-2H-quinazolin-3-yl]-methanesulfonamide;
A-13: N-[6-(1-Ethoxy-propyl)-2,4-dioxo-7-trifluoromethyl-1,4-dihydro-2H-quinazolin-3-yl]-methanesulfonamide;
A-14: N-[6-(1-Cyclopentyloxy-ethyl)-2,4-dioxo-7-trifluoromethyl-1,4-dihydro-2H-quinazolin-3-yl]-methanesulfonamide;
A-15: N-[6-(1-Hydroxy-butyl)-2,4-dioxo-7-trifluoromethyl-1,4-dihydro-2H-quinazolin-3-yl]-methanesulfonamide;
A-16: N-[6-(1-Methoxy-2-methyl-propyl)-2,4-dioxo-7-trifluoromethyl-1,4-dihydro-2H-quinazolin-3-yl]-methanesulfonamide;
A-17: N-[6-(3-Hydroxy-propyl)-2,4-dioxo-7-trifluoromethyl-1,4-dihydro-2H-quinazolin-3-yl]-methanesulfonamide;
A-18: N-[6-(1-Hydroxy-3-methoxy-propyl)-2,4-dioxo-7-trifluoromethyl-1,4-dihydro-2H-quinazolin-3-yl]-methanesulfonamide;
A-19: N-[6-(1-Hydroxy-2-methyl-propyl)-2,4-dioxo-7-trifluoromethyl-1,4-dihydro-2H-quinazolin-3-yl]-methanesulfonamide;
B-1: N-[2,4-Dioxo-6-(tetrahydro-pyran-2-yl)-7-trifluoromethyl-1,4-dihydro-2H-quinazolin-3-yl]-methanesulfonamide;
B-2: N-[2,4-Dioxo-6-(tetrahydro-furan-2-yl)-7-trifluoromethyl-1,4-dihydro-2H-quinazolin-3-yl]-methanesulfonamide;
B-3: N-[2,4-Dioxo-6-(tetrahydro-furan-3-yl)-7-trifluoromethyl-1,4-dihydro-2H-quinazolin-3-yl]-methanesulfonamide;
C-1: N-{7-isopropyl-6-[2-(2-methoxy-ethyl)-2H-pyrazol-3-yl]-2,4-dioxo-1,4-dihydro-2H-quinazolin-3-yl}-methanesulfonamide
C-2: N-[6-(2-Isopropyl-2H-pyrazol-3-yl)-2,4-dioxo-7-trifluoromethyl-1,4-dihydro-2H-quinazolin-3-yl]-methanesulfonamide;
C-3: N-[7-Fluoromethyl-6-(2-isopropyl-2H-pyrazol-3-yl)-2,4-dioxo-1,4-dihydro-2H-quinazolin-3-yl]-methanesulfonamide;
C-4: N-{6-[2-(2-Methoxy-ethyl)-2H-pyrazol-3-yl]-2,4-dioxo-7-trifluoromethyl-1,4-dihydro-2H-quinazolin-3-yl}-methanesulfonamide;
C-5: N-[6-(2-Hydroxy-2H-pyrazol-3-yl)-2,4-dioxo-7-trifluoromethyl-1,4-dihydro-2H-quinazolin-3-yl]-methanesulfonamide;
C-6: N-[7-Ethyl-6-(2-isopropyl-2H-pyrazol-3-yl)-2,4-dioxo-1,4-dihydro-2H-quinazolin-3-yl]-methanesulfonamide;
C-7: N-[7-Isopropyl-6-(2-methyl-2H-pyrazol-3-yl)-2,4-dioxo-1,4-dihydro-2H-quinazolin-3-yl]-methanesulfonamide;
C-8: N-[7-Isopropyl-6-(2-isopropyl-2H-pyrazol-3-yl)-2,4-dioxo-1,4-dihydro-2H-quinazolin-3-yl]-methanesulfonamide;
C-9: N-[7-Difluoromethyl-6-(2-methyl-2H-pyrazol-3-yl)-2,4-dioxo-1,4-dihydro-2H-quinazolin-3-yl]-methanesulfonamide;
C-10: N-[7-Difluoromethyl-6-(2-isopropyl-2H-pyrazol-3-yl)-2,4-dioxo-1,4-dihydro-2H-quinazolin-3-yl]-methanesulfonamide;
C-11: N-[7-Ethyl(2-methyl-2H-pyrazol-3-yl)-2,4-dioxo-1,4-dihydro-2H-quinazolin-3-yl]-methanesulfonamide,
C-12: N-[7-Ethyl-6-(2-ethyl-2H-pyrazol-3-yl)-2,4-dioxo-1,4-dihydro-2H-quinazolin-3-yl]-methanesulfonamide;
C-13: N-[7-Fluoromethyl-6-(2-methyl-2H-pyrazol-3-yl)-2,4-dioxo-1,4-dihydro-2H-quinazolin-3-yl]-methanesulfonamide;
C-14: N-[7-(1-fluoro-ethyl)-6-(2-methyl-2H-pyrazol-3-yl)-2,4-dioxo-1,4-dihydro-2H-quinazolin-3-yl]-methanesulfonamide;
C-15: N-[7-(1,1-difluoro-ethyl)-6-(2-methyl-2H-pyrazol-3-yl)-2,4-dioxo-1,4-dihydro-2H-quinazolin-3-yl]-methanesulfonamide;
C-16: N-[7-(1,1-difluoro-ethyl)-6-(2-isopropyl-2H-pyrazol-3-yl)-2,4-dioxo-1,4-dihydro-2H-quinazolin-3-yl]-methanesulfonamide;
C-17: N-[7-(1-fluoro-ethyl)-6-(2-isopropyl-2H-pyrazol-3-yl)-2,4-dioxo-1,4-dihydro-2H-quinazolin-3-yl]-methanesulfonamide; and
C-18: N-[6-(2-Methyl-2H-pyrazol-3-yl)-2,4-dioxo-7-trifluoromethyl-1,4-dihydro-2H-quinazolin-3-yl]-methanesulfonamide.

In one embodiment of the invention, the 1H-quinazoline-2,4-dione of formula (I) is a compound selected from the group consisting of compound A-1, A-2, A-3, A-4, A-5, A-6, A-7, A-8, A-9, A-10, A-11, A-12, A-13, A-14, A-15, A16, A17, A-18 and A-19.

In one embodiment of the invention, the 1H-quinazoline-2,4-dione of formula (I) is a compound selected from the group consisting of compound B-1, B-2 and B-3.

In one embodiment of the invention, the 1H-quinazoline-2,4-dione of formula (I) is a compound selected from the group consisting of compound C-1, C-2, C-3, C-4, C-5, C-6, C-7, C-8, C-9, C-10, C-11, C-12, C-13, C-14, C-15, C-16, C-17 and C-18.

Advantageous compounds of the invention, i.e., the 1H-quinazoline-2,4-diones of formula (I), should be well absorbed from the gastrointestinal tract, penetrate the blood brain barrier, be sufficiently metabolically stable and possess favorable pharmacokinetic properties.

Preferred compounds, having superior bioavailibility are 1H-quinazoline-2,4-dione of formula (I) selected from the group consisting of compounds: A-1, A-2, A-3, A-4, A-5, A-6, A-7, A-13, A-14, A-15, A-18, B-2, B-3, C-1, C-2, C-3, C-4, C-5, C-6, C-7, C-8, C-9, C-10, C-11, C-12, C-15, C-16, C-17 and C-18.

More preferred compounds, having superior bioavailibility are 1H-quinazoline-2,4-dione of formula (I) selected from the group consisting of compounds: A-1, A-2, A-3. A-4. A-5, A-7, A-15, B-2, B-3, C-1, C-2, C-3, C-6, C-7, C-8, C-9, C-10, C-11, C-12, C-15, C-17 and C-18.

Further more preferred compounds, having superior bioavailibility are 1H-quinazoline-2,4-dione of formula (I) selected from the group consisting of compounds: A-2. A-3, A-4, A-5 B-2, C-2, C-3, C-7, C-9, C-10, C-11, C-15 and C-18.

Most preferred compounds, having superior bioavailibility are 1H-quinazoline-2,4-dione of formula (I) selected from the group consisting of compounds: A-2, A-5, B-2, C-7, C-9 and C-11 .

Preferred prodrugs of the invention should be well absorbed from the gastrointestinal tract, be transformed into the parent compound (or active principle, being the compound that in-vivo acts as AMPA receptor antagonist), the parent compound should be sufficiently metabolically stable and possess favorable pharmacokinetic properties.

Further preferred prodrugs of the invention lead to an oral bioavailability of the parent compound which is comparable to the bioavailability when administered as a drug.

Further preferred prodrugs of the invention exhibit increased oral bioavailability compared to the parent compound when administered as a drug. Oral bioavailability may manifest itself in different ways: (i) a biological effect may be achieved after oral administration when the parent compound is less effective upon oral administration, (ii) an earlier onset of action upon oral administration, (iii) a lower dose needed to achieve the same effect, (iv) a higher effect achieved by the same dose or (v) a prolonged action at the same dose.

Further preferred prodrugs of the invention are transformed into parent compounds which in-vivo bind potently to AMPA receptors whilst showing little affinity for other receptors.

Some prodrugs of the invention are transformed into parent compounds which also show antagonistic activity at kainate receptors. As migraine is a condition where an overactivity of kainate receptors is implicated, said prodrugs are suitable to treat migraine. Besides such dual activity, showing little affinity for other receptors is a preferred feature.

Further prodrugs of the invention—when the active principle is targeted against receptors in the central nervous system—are transformed into parent compounds that cross the blood brain barrier freely.

Further prodrugs of the invention—when the active principle is targeted selectively against receptors in the peripheral nervous system—are transformed into parent compounds that do not cross the blood brain barrier.

Prodrugs, parent compounds and released pro-moieties should be non-toxic and demonstrate few side-effects.

Furthermore, the ideal prodrug of the invention will be able to exist in a physical form that is stable, non-hygroscopic and easily formulated.

The higher oral bioavailability of the compounds for use in the invention may give rise to the following beneficial effects relating to less bioavailable compounds: (i) an enhanced biological effect may be achieved after oral administration: (ii) an earlier onset of action may be observed following oral administration; (iii) a lower dose may be needed to achieve the same effect; (iv) a higher effect may be achieved by the same dose or (v) a prolonged action may be observed at the same dose.

Preferably the compound for use in the invention when tested in-vivo potently binds to AMPA receptors whilst showing little affinity for other receptors.

In the present specification, the following definitions shall apply if no specific other definition is given:

The term “neuronal ceroid lipofuscinosis” (NCL) is used for a group of autosomal recessively inherited lysosomal storage disorders characterized by progressive neurodegeneration (J D Cooper, Current Opinion in Neurology, 16, 121-128, 2003). NCL is typified by its progressive nature, presenting with visual disturbances leading to blindness, progressing cerebral dysfunctions, such as cognitive and motor dysfunctions, an increased severity of untreatable seizures and ultimately premature death. The vast majority of cases manifest during childhood with an infantile, late infantile or juvenile onset, although rare adult forms and variant forms are also recognized NCL is considered the most common pediatric neurodegenerative disease, with a global incidence of 1-8 in 100000 births (N Zhong, Mol Genet Metab, 71, 195-206, 2000). As yet, no specific treatment is known that can slow the progress or even halt the disease.

So far, eight genetically distinct forms of NCL have been identified: Infantile NCL (INCL, Santavuori-Haltia disease, linked to mutations in the CLN1 gene), Late infantile NCL (LINCL, Jansky-Bielschowsky disease, linked to mutations in the CLN2 gene), juvenile NCL (JNCL, Batten disease, linked to mutations in the CLN3 gene), Adult NCL (ANCL, Kufs disease, Parry's disease, linked to mutations in the CLN4 gene), Finnish Late Infantile NCL (fLINCL, linked to mutations in the CLN5 gene), Portuguese Late Infantile NCL (pLINCL, linked to mutations in the CLN6 gene), Turkish Late Infantile NCL (tLINCL, linked to mutations in the CLN7 gene) and Progressive Epilepsy with Mental Retardation (EPMR, “nothern epilepsy”, linked to mutations in the CLN8 gene).

The most prevalent form of NCL is the juvenile form, also called Batten disease.

In one embodiment, the neuronal ceroid lipofuscinosis is Batten disease.

In one embodiment, the neuronal ceroid lipofuscinosis is Infantile NCL.

In one embodiment, the neuronal ceroid lipofuscinosis is Late infantile NCL.

In one embodiment, the neuronal ceroid lipofuscinosis is Adult NCL.

In one embodiment, the neuronal ceroid lipofuscinosis is Finnish Late Infantile NCL.

In one embodiment, the neuronal ceroid lipofuscinosis is Portuguese Late Infantile NCL.

In one embodiment, the neuronal ceroid lipofuscinosis is Turkish Late Infantile NCL.

In one embodiment, the neuronal ceroid lipofuscinosis is Progressive Epilepsy with Mental Retardation.

The term “subject” as used herein refers to a human or non-human being, preferably a human, especially to a patient being diagnosed with neuronal ceroid lipofuscinosis.

The term “treatment” as used herein is intended to mean administration or application of the medicament containing 1H-quinazoline-2,4-diones of formula (I) to a patient affected by neuronal ceroid lipofuscinosis and related conditions. The term “treatment” as used herein refers to any type of treatment that imparts a benefit to a subject affected with a disease, e.g. a patient diagnosed with a disease, including improvement in the condition of the subject (e.g. in one or more symptoms), delay in the progression of the disease etc. Treatment typically comprise a reduction in the symptoms associated with neuronal ceroid lipofuscinoses, including for example, although not limited to, a reduction in visual disturbances in an early stage of the disease, a reduction in neurocognitive and/or motor function decline or a reduction in number and severity of seizures.

The term “therapeutically effective amount” as used herein typically refers to a drug amount which, when administered to a subject, is sufficient to provide a therapeutic benefit, e.g. is sufficient for treating, preventing or delaying the progression of neuronal ceroid lipofuscinoses (e.g. the amount provides an amelioration of symptoms, e.g. it leads to a reduction in number and severity of seizures).

The term “prevention” is intended to mean administration or application of the medicament containing 1H-quinazoline-2,4-diones of formula (I) to a patient in order to prevent the onset of neuronal ceroid lipofuscinoses and related conditions, e.g. administration or application of the medicament shortly to a patient predicted to be at risk of developing neuronal ceroid lipofuscinoses.

The term “delay of progression” is intended to mean administration or application of the medicament containing 1H-quinazoline-2,4-diones of formula (I) to a patient in order to postpone the progression of neuronal ceroid lipofuscinoses and related conditions.

For the above-mentioned indications (the conditions and disorders) the appropriate dosage will vary depending upon, for example, the compound employed, the host, the mode of administration and the nature and severity of the condition being treated. However, in general, satisfactory results in animals are indicated to be obtained at a daily dosage of from about 0.01 to about 100 mg/kg body weight, preferably from about 1 to about 30 mg/kg body weight, e.g. 10 mg/kg. In larger mammals, for example humans, an indicated daily dosage is in the range from about 0.1 to about 1000 mg, preferably from about 1 to about 400 mg, most preferably from about 10 to about 100 mg of a 1H-quinazoline-2,4-dione of formula (I) conveniently administered, for example, in divided doses up to four times a day.

For use according to the invention, the 1H-quinazoline-2,4-diones of formula (I) may be administered as single active agent or in combination with other active agents, in any usual manner, e.g. orally, for example in the form of tablets, capsules or drinking solutions; rectally, for example in the form of suppositories; intravenous, for example in the form of injection solutions or suspensions; or transdermally, for example in the form of a patch.

In one embodiment, the manner of administration is oral administration, for example in the form of a tablet, capsule or drinking solution.

In one embodiment, the manner of administration is rectal administration, for example in the form of a suppository.

In one embodiment, the manner of administration is transdermal administration, for example in the form of a patch.

Moreover, the present invention provides a pharmaceutical composition comprising a 1H-quinazoline-2,4-diones of formula (I) in association with at least one pharmaceutical carrier or diluent for the treatment, prevention or delay of progression of neuronal ceroid lipofuscinosis. Such compositions may be manufactured in conventional manner. Unit dosage forms may contain, for example, from about 2.5 to about 250 mg, preferably from about 2.5 to about 200 mg, more preferably from about 2.5 to about 100 mg, still more preferably from about 2.5 to about 50 mg and still more preferably from about 2.5 to about mg, of one or more of the 1H-quinazoline-2,4-diones of formula (I).

The pharmaceutical compositions according to the invention are compositions for enteral administration, such as oral or rectal administration; or parenteral administration, such as intramuscular, intravenous, nasal or transdermal administration, to warm-blooded animals (human beings and animals) that comprise an effective dose of the pharmacological active ingredient alone or together with a significant amount of a pharmaceutically acceptable carrier. The dose of the active ingredient depends on the species of warm-blooded animal, body weight, age and individual condition, individual pharmacokinetic data, the disease to be treated and the mode of administration.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

The pharmaceutical compositions of the present invention are prepared in a manner known per se, for example by means of conventional dissolving, lyophilizing, mixing, granulating or confectioning processes. Such processes are exemplified in WO 2005/079802, WO 2003/047581, WO 2004/000316, WO 2005/044265. WO 2005/044266, WO 2005/044267, WO 2006/114262 and WO 2007/071358.

Compositions for transdermal are described in Remington's Pharmaceutical Sciences 16^{th} Edition Mack; Sucker, Fuchs and Spieser. Pharmazeutische Technologie, 1^{st} Edition, Springer.

Efficacy of the compounds of the invention in the treatment of neuronal ceroid lipofuscinoses and related conditions may be demonstrated by any suitable in vitro or in vivo testing procedure. For example, the efficacy may be demonstrated using the following procedures.

### Oral Bioavailability of the Compounds of the Invention

Oral bioavailability of the compounds of the invention may be demonstrated using any generally known test in which the compound is administered orally and a biological effect observed.

Oral bioavailability of the compounds of the invention in the treatment of neuronal ceroid lipofuscinoses may be further quantified by the Maximal Electroshock test, which demonstrates that the compounds are orally bioavailable, penetrate the blood brain barrier and bind to the target receptor.

The oral bioavailability was tested using the audiogenic mouse test (Audiogenic seizures, R. L. Collins; Chapter 14, pages: 347-372. In: Experimental Models of Epilepsy; By: Pupura, Penry, Tower, Woodbury, Waiter, Raven Press, New York, 1972. Standard Book Number: 0-911216-26-X) and/or the MES test. Where the MES test was used (as described below), the result is given in Table 1.

### In-Vivo Activity of Parent Compounds and Prodrugs in the Murine Maximal Electro Shock Test

Compounds of the invention were tested in OF1 mice using the maximal electroshock test (MES Test) described in detail by Schmutz et al., Naunyn-Schmiedeberg's Arch Pharmacol 1990, 342, 61-66. Briefly, generalized tonic-clonic convulsions of the hind extremities were induced by passing electrical current through temporal electrodes (50 Hz, 18 mA, 0.2 s). Mice treated by vehicle showed mean seizure durations of 12-14 s. 30 mg/kg carbamazepine was used as a positive control; mice were classified as protected by a compound if the duration of the seizure lasted only 3 second or less. Five mice were used for each treatment condition and the percentage of protected mice was used as readout (i.e. a compound could give 0%, 20%, 40%, 60%, 80% or 100% protection). Compounds of the invention were given at a dose of 50 mg/kg, p.o., 1 hour prior to induction of convulsions (i.e. “pre-treatment time−1 h”).

ED50 values (ED: effective dose) were calculated using GraphPad Prism, v4.02.

15 s after shock administration, mouse blood was collected for determination of compounds' blood exposure.

The results are shown below in Table 1.

TABLE 1

| | | In vivo | MES-Test | |
|---|---|---|---|---|
| | | orally | (1 h, po) | |
| Compound | Structure | active | ED50 [mg/kg] | IUPAC name |
| A-1 | 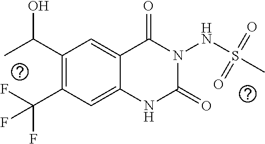 | Yes | 64 | N-[6-(1-Hydroxy-ethyl)-2,4-dioxo-7- trifluoromethyl-1,4-dihydro-2H-quinazolin-3-yl]- methanesulfonamide |
| A-2 | 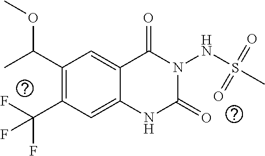 | Yes | 6.0 | N-[6-(1-Methoxy-ethyl)-2,4-dioxo-7- trifluoromethyl-1,4-dihydro-2H-quinazolin-3-yl]- methanesulfonamide |
| A-3 | 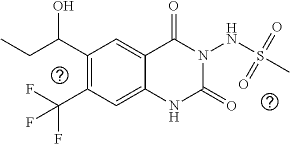 | Yes | 19.6 | N-[6-(1-Hydroxy-propyl)-2,4-dioxo-7- trifluoromethyl-1,4-dihydro-2H-quinazolin-3-yl]- methanesulfonamide |
| A-4 | 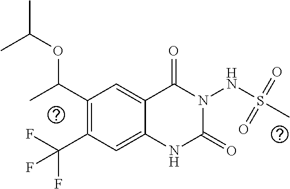 | Yes | 15.6 | N-[6-(1-Isopropoxy-ethyl)-2,4-dioxo-7- trifluoromethyl-1,4-dihydro-2H-quinazolin-3-yl]- methanesulfonamide |
| A-5 | 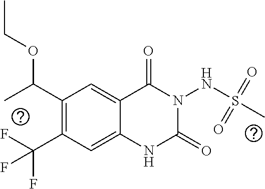 | Yes | 8.8 | N-[6-(1-Ethoxy-ethyl)-2,4-dioxo-7- trifluoromethyl-1,4-dihydro-2H-quinazolin-3-yl]- methanesulfonamide |
| A-6 | 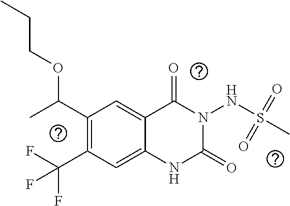 | Yes | nt¹ | N-[2,4-Dioxo-6-(1-propoxy-propyl)-7- trifluoromethyl-1,4-dihydro-2H-quinazolin-3-yl]- methanesulfonamide |
| A-7 | 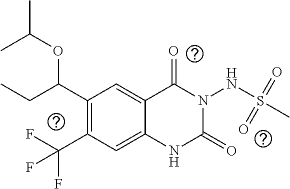 | Yes | 24.7 | N-[6-(1-Isopropoxy-propyl)-2,4-dioxo-7- trifluoromethyl-1,4-dihydro-2H-quinazolin-3-yl]- methanesulfonamide |
| A-8 | 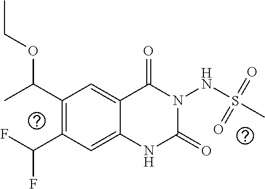 | nt | nt | N-[7-Difluoromethyl-6-(1-ethoxy-ethyl)-2,4- dioxo-1,4-dihydro-2H-quinazolin-3-yl]- methanesulfonamide |
| A-9 | 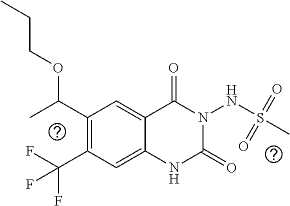 | nt | nt | N-[2,4-Dioxo-6-(1-propoxy-ethyl)-7- trifluoromethyl-1,4-dihydro-2H-quinazolin-3-yl]- methanesulfonamide |
| A-10 | 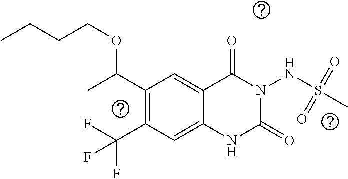 | nt | nt | N-[6-(1-Butoxy-ethyl)-2,4-dioxo-7- trifluoromethyl-1,4-dihydro-2H-quinazolin-3-yl]- methanesulfonamide |
| A-11 | 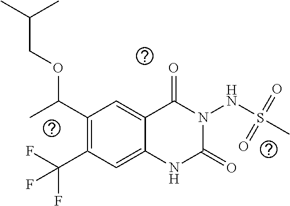 | nt | nt | N-[6-(1-Isobutoxy-ethyl)-2,4-dioxo-7- trifluoromethyl-1,4-dihydro-2H-quinazolin-3-yl]- methanesulfonamide |
| A-12 | 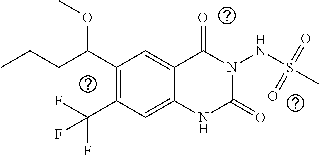 | nt | nt | N-[6-(1-methoxy-butyl)-2,4-dioxo-7- trifluoromethyl-1,4-dihydro-2H-quinazolin-3-yl]- methanesulfonamide |
| A-13 | 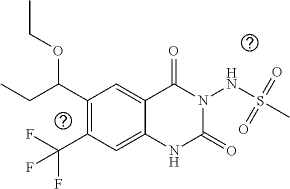 | Yes | nt | N-[6-(1-Ethoxy-propyl)-2,4-dioxo-7- trifluoromethyl-1,4-dihydro-2H-quinazolin-3-yl]- methanesulfonamide |
| A-14 | 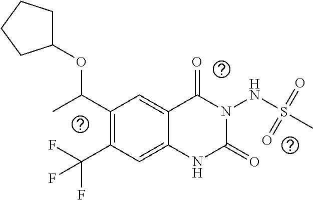 | yes | nt | N-[6-(1-Cyclopentyloxy-ethyl)-2,4-dioxo-7- trifluoromethyl-1,4-dihydro-2H-quinazolin-3-yl]- methanesulfonamide |
| A-15 | 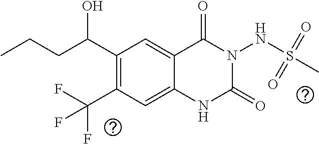 | Yes | 35 | N-[6-(1-Hydroxy-butyl)-2,4-dioxo-7- trifluoromethyl-1,4-dihydro-2H-quinazolin-3-yl]- methanesulfonamide |
| A-16 | 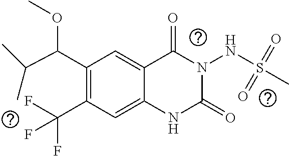 | nt | nt | N-[6-(1-Methoxy-2-methyl-propyl)-2,4-dioxo-7- trifluoromethyl-1,4-dihydro-2H-quinazolin-3-yl]- methanesulfonamide |
| A-17 | 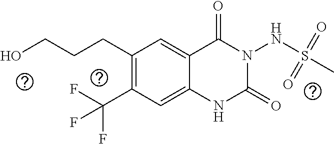 | nt | nt | N-[6-(3-Hydroxy-propyl)-2,4-dioxo-7- trifluoromethyl-1,4-dihydro-2H-quinazolin-3-yl]- methanesulfonamide |
| A-18 | 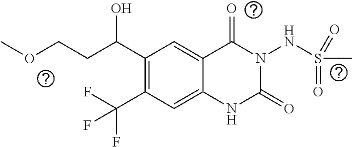 | Yes | nt | N-[6-(1-Hydroxy-3-methoxy-propyl)-2,4-dioxo-7- trifluoromethyl-1,4-dihydro-2H-quinazolin-3-yl]- methanesulfonamide |
| A-19 | 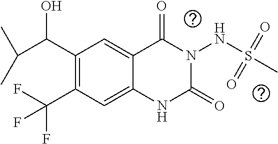 | nt | nt | N-[6-(1-Hydroxy-2-methyl-propyl)-2,4-dioxo-7- trifluoromethyl-1,4-dihydro-2H-quinazolin-3-yl]- methanesulfonamide |
| B-1 | 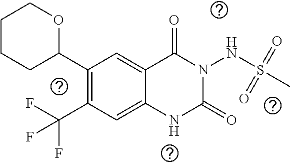 | nt | nt | N-[2,4-Dioxo-6-(tetrahydro-pyran-2-yl)-7- trifluoromethyl-1,4-dihydro-2H-quinazolin-3-yl]- methanesulfonamide |
| B-2 | 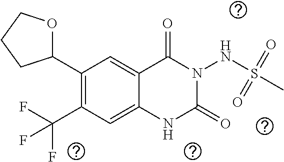 | Yes | 12.8 (R)² 33.2 (S) | N-[2,4-Dioxo-6-(tetrahydro-furan-2-yl)-7- trifluoromethyl-1,4-dihydro-2H-quinazolin-3-yl]- methanesulfonamide |
| B-3 | 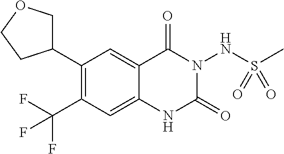 | Yes | 20% @ 25³ | N-[2,4-Dioxo-6-(tetrahydro-furan-3-yl)-7- trifluoromethyl-1,4-dihydro-2H- quinazolin-3-yl]-methanesulfonamide |
| C-1 | 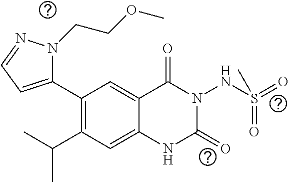 | yes | 40% @ 25 | N-{7-Isopropyl-6-[2-(2-methoxy-ethyl)-2H- pyrazol-3-yl]-2,4-dioxo-1,4-dihydro-2H- quinazolin-3-yl}-methanesulfonamide |
| C-2 | 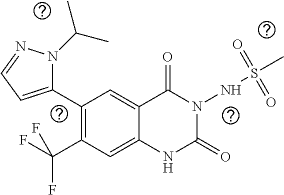 | Yes | 17.7 | N-[6-(2-Isopropyl-2H-pyrazol-3-yl)-2,4-dioxo-7- trifluoromethyl-1,4-dihydro-2H-quinazolin-3-yl]- methanesulfonamide |
| C-3 | 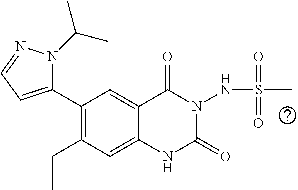 | Yes | 13.5 | N-[7-Fluoromethyl-6-(2-isopropyl-2H-pyrazol-3- yl)-2,4-dioxo-1,4-dihydro-2H-quinazolin-3-yl]- methanesulfonamide |
| C-4 | 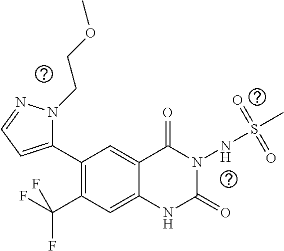 | Yes | nt | N-{6-[2-(2-Methoxy-ethyl)-2H-pyrazol-3-yl]-2,4- dioxo-7-trifluoromethyl-1,4-dihydro-2H- quinazolin-3-yl}-methanesulfonamide |
| C-5 | 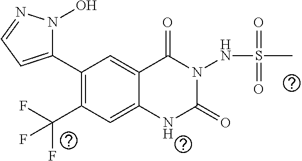 | Yes | nt | N-[6-(2-Hydroxy-2H-pyrazol-3-yl)-2,4-dioxo-7- trifluoromethyl-1,4-dihydro-2H-quinazolin-3-yl]- methanesulfonamide |
| C-6 | 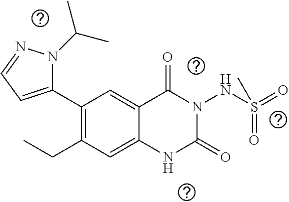 | yes | 20% @ 50 | N-[7-Ethyl-6-(2-isopropyl-2H-pyrazol-3-yl)-2,4- dioxo-1,4-dihydro-2H-quinazolin-3-yl]- methanesulfonamide |
| C-7 | 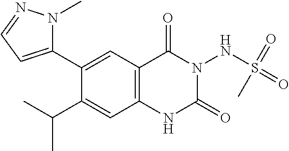 | Yes | 6.9 | N-[7-Isopropyl-6-(2-methyl-2H-pyrazol-3-yl)- 2,4-dioxo-1,4-dihydro-2H-quinazolin-3-yl]- methanesulfonamide |
| C-8 | 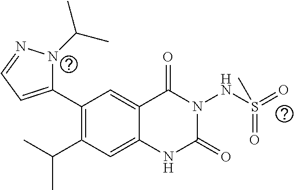 | yes | 40% @ 50 | N-[7-Isopropyl-6-(2-isopropyl-2H-pyrazol-3-yl)- 2,4-dioxo-1,4-dihydro-2H-quinazolin-3-yl]- methanesulfonamide |
| C-9 | 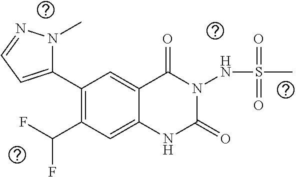 | Yes | 7.5 | N-[7-Difluoromethyl-6-(2-methyl-2H-pyrazol-3- yl)-2,4-dioxo-1,4-dihydro-2H-quinazolin-3-yl]- methanesulfonamide |
| C-10 | 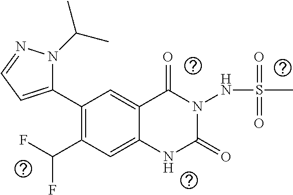 | Yes | 20.3 | N-[7-Difluoromethyl-6-(2-isopropyl-2H-pyrazol- 3-yl)-2,4-dioxo-1,4-dihydro-2H-quinazolin-3-yl]- methanesulfonamide |
| C-11 | 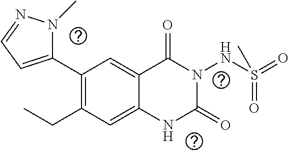 | Yes | 6.1 | N-[7-Ethyl-6-(2-methyl-2H-pyrazol-3-yl)-2,4- dioxo-1,4-dihydro-2H-quinazolin-3-yl]- methanesulfonamide |
| C-12 | 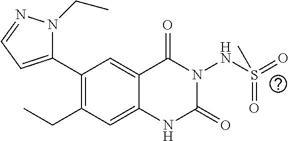 | Yes | 42.8 | N-[7-Ethyl-6-(2-ethyl-2H-pyrazol-3-yl)-2,4- dioxo-1,4-dihydro-2H-quinazolin-3-yl]- methanesulfonamide |
| C-13 | 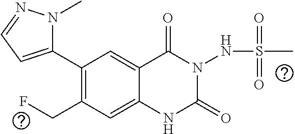 | nt | nt | N-[7-Fluoromethyl-6-(2-methyl-2H-pyrazol-3- yl)-2,4-dioxo-1,4-dihydro-2H-quinazolin-3-yl]- methanesulfonamide |
| C-14 | 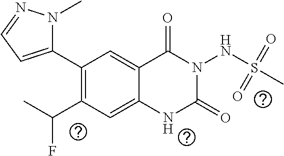 | nt | nt | N-[7-(1-fluoro-ethyl)-6-(2-methyl-2H-pyrazol-3- yl)-2,4-dioxo-1,4-dihydro-2H-quinazolin-3-yl]- methanesulfonamide |
| C-15 | 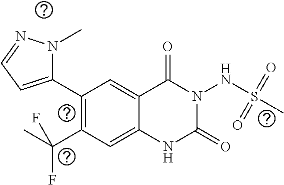 | Yes | 80% @ 20 | N-[7-(1,1-difluoro-ethyl)-6-(2-methyl-2H- pyrazol-3-yl)-2,4-dioxo-1,4-dihydro-2H- quinazolin-3-yl]-methanesulfonamide |
| C-16 | 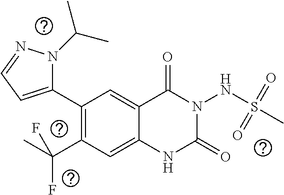 | Yes | nt | N-[7-(1,1-difluoro-ethyl)-6-(2-isopropyl-2H- pyrazol-3-yl)-2,4-dioxo-1,4-dihydro-2H- quinazolin-3-yl]-methanesulfonamide |
| C-17 | 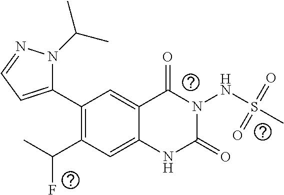 | Yes | >20 | N-[7-(1-fluoro-ethyl)-6-(2-isopropyl-2H-pyrazol- 3-yl)-2,4-dioxo-1,4-dihydro-2H-quinazolin-3-yl]- methanesulfonamide |
| C-18 | 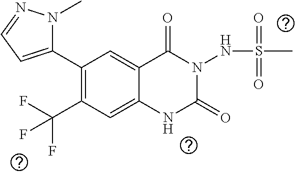 | Yes | 14.8 | N-[6-(2-Methyl-2H-pyrazol-3-yl)-2,4-dioxo-7- trifluoromethyl-1,4-dihydro-2H-quinazolin-3-yl]- methanesulfonamide |
| Comparative | 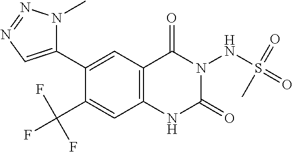 | No | 0% @ 50 | N-(6-(1-methyl-1H-1,2,3-triazol-5-yl)-2,4-dioxo- 7-trifluoromethyl-1,4-dihydro-2H-quinazolin-3- yl]-methanesulfonamide |

¹The term “nt” through out the table means “not tested”
²(R) and (S) indicate the two enantiomers.
³The term “20% @ 25” means 20% protection at 50 mg/kg.

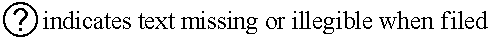

This data shows that the compounds for use in the invention exhibit beneficial oral bioavailability relating to the comparative example (not in accordance with the invention).

### 1. Diagnosis

Methods for diagnosing neuronal ceroid lipofuscinosis are known. Diagnosis of NCL is based on age of onset, clinico-pathological findings, microscopic analysts of autofluorescent lysosomal storage material and mutation analysis of the underlying genes. The vast majority of cases manifest during childhood with infantile, late infantile or juvenile onset, although rare adult forms are also recognized. Most patients with NCL have progressive ocular and cerebral dysfunction, including cognitive and motor dysfunction and uncontrolled seizures, finally resulting in wheelchair or bedridden stages and premature death. Genotyping of the eight NCL forms (CLN1-CLN8) provides the definitive diagnosis and has thus far identified over 150 mutations.

The usefulness of the 1H-quinazoline-2,4-diones of formula (I) in the treatment of the above-mentioned disorders can be confirmed in a range of standard tests including those indicated below.

2. Assessment of Anti-Ataxic Effect of 1H-quinazoline-2,4-diones of Formula (I) in Mutant Mice

### 2.1 Method

Mice with a targeted disruption of the CLN3 gene are considered a valid mouse model for Batten disease (Mitchison et al, Neurobiology of Disease, 6, 321-334, 1999), Homozygous mice with a targeted mutation of the CLN3 gene (Jackson Laboratory, mouse strain number 004685) display with age an increasing motor performance deficit.

### 2.2 Assessment

Motor performance is quantified by the ability to remain in position on a rotating rod (“rota-rod” test). Mice obtaining vehicle controls and acute/repeated dosing of 1H-quinazoline-2,4-diones of formula (I) are analyzed.

### 2.3 Protocol

Mice are tested at different ages. One group of mice is treated with an effective dose of 1H-quinazoline-2,4-dione of formula (I), the control group receives the vehicle only. The 1H-quinazoline-2,4-dione compounds of formula (I) are tested for motor coordination on the rotating rod acutely and after repeated dosing.

### 3. Clinical Testing: Improvement Trials

Characteristics/Symptoms of neuronal ceroid lipofuscinosis are described above and include visual disturbances, neurocognitive and motor function decline and an increased severity of seizures. The improvement of such deficits can be measured in clinical trials. Clinical testing of the 1H-quinazoline-2,4-diones of formula (I) may be conducted, for example, in one of the following study designs. The skilled physician may look at a number of aspects of patient behaviours and abilities. He will realise that such studies are considered as guidelines and the certain aspects of the studies may be modified and redefined depending on the circumstance and environment, for example.

### 3.1 Trial A: Normal Patient Population

A patient population, with a normal control is dosed once a day for a week or longer tested. The test is designed to allow for improvement, i.e. that there is a measurable parameter increase of the impaired function The patients are tested at the beginning and at the end of the dosage period and the results are compared and analyzed.

### 3.2 Trial B: Deficit Population

A patient population with a deficit associated with neuronal ceroid lipofuscinosis is dosed once a day for a week or longer and tested. The test is designed to allow for improvement, i.e. that there is a measurable parameter increase of the impaired function. The patients are tested at the beginning and at the end of the dosage period and the results are compared and analyzed. Exemplary parameters to test could include fewer or absence of seizures, improved visual performance or restored memory-dependent or motor functions. Also measurable could be visualization of the reversal of some of the neuronal structural defects (by imaging).

### 3.3 Considerations for Design a Trial

When designing a trial, the skilled person will appreciate the need to protect both against floor and ceiling effects. In other words the study designing should allow cognition to the measurably raised or lowered.
Conditions that artificially impair a function, e.g. cognition, are one way to test enhancement of that function. Such conditions are for example, sleep deprivation and pharmacological challenges.
Placebo control is required for all trials.
In assessing the data evaluation of the likelihood of learning and practice effects from repeat assessments must be made. The likelihood of such effects contaminating the data to produce false positives should be taken in to account when designing the test, e.g. the tests should not be identical (e.g. commit the same list of words to memory) but designed to study the same mechanism. Other countermeasures may include single testing at the end of a trial only

1. A compound of formula (I); 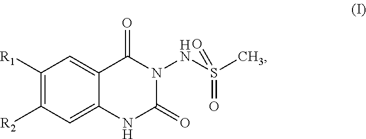
wherein
R₁ is C₁-C₆alkyl substituted by one, two or three substituents selected from hydroxy, C₁-C₆alkoxy or C₅-C₆cycloalkoxy; C₅-C₆cycloalkyl substituted by one, two or three substituents selected from hydroxy, C₁-C₆alkoxy or C₅-C₆cycloalkoxy; or
R₁ is 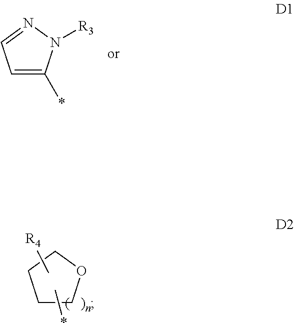
R₃ is C₁-C₆alkyl, hydroxy or C₁-C₆alkoxy-C₁-C₆alkyl;
R₄ is hydrogen or C₁-C₆alkyl;
n is 1 or 2;
R₂ is C₁-C₃alkyl or C₁-C₃fluoroalkyl;
and their pharmaceutically acceptable salts and prodrugs thereof;
for the treatment, prevention or delay of progression of neuronal ceroid lipofuscinosis.

2. A compound of formula (I) according to claim 1, wherein R₁ is D1 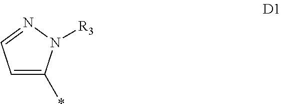
R₃ is C₁-C₆alkyl, hydroxy or C₁-C₆alkoxy-C₁-C₆alkyl;
R₂ is C₁-C₃alkyl or C₁-C₃fluoroalkyl;
and their pharmaceutically acceptable salts and prodrugs thereof;
for the treatment, prevention or delay of progression of neuronal ceroid lipofuscinosis.

3. A compound of formula (I) according to claim 1, wherein R₁ is D2 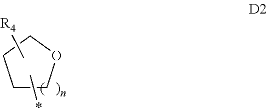
R₄ is hydrogen or C₁-C₆alkyl;
n is 1 or 2;
R₂ is C₁-C₃alkyl or C₁-C₃fluoroalkyl;
and their pharmaceutically acceptable salts and prodrugs thereof;
for the treatment, prevention or delay of progression of neuronal ceroid lipofuscinosis.

4. A compound of formula (I), their pharmaceutically acceptable salts and prodrugs thereof, according to claim 1, selected from the group consisting of:
N-[6-(1-Hydroxy-ethyl)-2,4-dioxo-7-trifluoromethyl-1,4-dihydro-2H-quinazolin-3-yl]-methanesulfonamide;
N-[6-(1-Methoxy-ethyl)-2,4-dioxo-7-trifluoromethyl-1,4-dihydro-2H-quinazolin-3-yl]-methanesulfonamide;
N-[6-(1-Hydroxy-propyl)-2,4-dioxo-7-trifluoromethyl-1,4-dihydro-2H-quinazolin-3-yl]-methanesulfonamide;
N-[6-(1-Isopropoxy-ethyl)-2,4-dioxo-7-trifluoromethyl-1,4-dihydro-2H-quinazolin-3-yl]-methanesulfonamide;
N-[6-(1-Ethoxy-ethyl)-2,4-dioxo-7-trifluoromethyl-1,4-dihydro-2H-quinazolin-3-yl]-methanesulfonamide;
N-[2,4-Dioxo-6-(1-propoxy-propyl)-7-trifluoromethyl-1,4-dihydro-2H-quinazolin-3-yl]-methanesulfonamide;
N-[6-(1-isopropoxy-propyl)-2,4-dioxo-7-trifluoromethyl-1,4-dihydro-2H-quinazolin-3-yl]-methanesulfonamide;
N-[7-Difluoromethyl-6-(1-ethoxy-ethyl)-2,4-dioxo-1,4-dihydro-2H-quinazolin-3-yl]-methanesulfonamide;
N-[2,4-Dioxo-6-(1-propoxy-ethyl)-7-trifluoromethyl-1,4-dihydro-2H-quinazolin-3-yl]-methanesulfonamide;
N-[6-(1-Butoxy-ethyl)-2,4-dioxo-7-trifluoromethyl-1,4-dihydro-2H-quinazolin-3-yl]-methanesulfonamide;
N-[6-(1-Isobutoxy-ethyl)-2,4-dioxo-7-trifluoromethyl-1,4-dihydro-2H-quinazolin-3-yl]-methanesulfonamide;
N-[6-(1-methoxy-butyl)-2,4-dioxo-7-trifluoromethyl-1,4-dihydro-2H-quinazolin-3-yl]-methanesulfonamide;
N-[6-(1-Ethoxy-propyl)-2,4-dioxo-7-trifluoromethyl-1,4-dihydro-2H-quinazolin-3-yl]-methanesulfonamide;
N-[6-(1-Cyclopentyloxy-ethyl)-2,4-dioxo-7-trifluoromethyl-1,4-dihydro-2H-quinazolin-3-yl]-methanesulfonamide;
N-[6-(1-Hydroxy-butyl)-2,4-dioxo-7-trifluoromethyl-1,4-dihydro-2H-quinazolin-3-yl]-methanesulfonamide;
N-[6-(1-Methoxy-2-methyl-propyl)-2,4-dioxo-7-trifluoromethyl-1,4-dihydro-2H-quinazolin-3-yl]-methanesulfonamide;
N-[6-(3-Hydroxy-propyl)-2,4-dioxo-7-trifluoromethyl-1,4-dihydro-2H-quinazolin-3-yl]-methanesulfonamide;
N-[6-(1-Hydroxy-3-methoxy-propyl)-2,4-dioxo-7-trifluoromethyl-1,4-dihydro-2H-quinazolin-3-yl]-methanesulfonamide;
N-[6-(1-Hydroxy-2-methyl-propyl)-2,4-dioxo-7-trifluoromethyl-1,4-dihydro-2H-quinazolin-3-yl]-methanesulfonamide;
N-[2,4-Dioxo-6-(tetrahydro-pyran-2-yl)-7-trifluoromethyl-1,4-dihydro-2H-quinazolin-3-yl]-methanesulfonamide;
N-[2,4-Dioxo-6-(tetrahydro-furan-2-yl)-7-trifluoromethyl-1,4-dihydro-2H-quinazolin-3-yl]-methanesulfonamide;
N-[2,4-Dioxo-6-(tetrahydro-furan-3-yl)-7-trifluoromethyl-1,4-dihydro-2H-quinazolin-3-yl]-methanesulfonamide;
N-{7-Isopropyl-6-[2-(2-methoxy-ethyl)-2H-pyrazol-3-yl]-2,4-dioxo-1,4-dihydro-2H-quinazolin-3-yl}-methanesulfonamide;
N-[6-(2-Isopropyl-2H-pyrazol-3-yl)-2,4-dioxo-7-trifluoromethyl-1,4-dihydro-2H-quinazolin-3-yl]-methanesulfonamide;
N-[7-Fluoromethyl-6-(2-isopropyl-2H-pyrazol-3-yl)-2,4-dioxo-1,4-dihydro-2H-quinazolin-3-yl]-methanesulfonamide;
N-{6-[2-(2-Methoxy-ethyl)-2H-pyrazol-3-yl]-2,4-dioxo-7-trifluoromethyl-1,4-dihydro-2H-quinazolin-3-yl}-methanesulfonamide;
N-[6-(2-Hydroxy-2H-pyrazol-3-yl)-2,4-dioxo-7-trifluoromethyl-1,4-dihydro-2H-quinazolin-3-yl]-methanesulfonamide;
N-[7-Ethyl-6-(2-isopropyl-2H-pyrazol-3-yl)-2,4-dioxo-1,4-dihydro-2H-quinazolin-3-yl]-methanesulfonamide;
N-[7-Isopropyl-6-(2-methyl-2H-pyrazol-3-yl)-2,4-dioxo-1,4-dihydro-2H-quinazolin-3-yl]-methanesulfonamide;
N-[7-Isopropyl-6-(2-isopropyl-2H-pyrazol-3-yl)-2,4-dioxo-1,4-dihydro-2H-quinazolin-3-yl]-methanesulfonamide;
N-[7-Difluoromethyl-6-(2-methyl-2H-pyrazol-3-yl)-2,4-dioxo-1,4-dihydro-2H-quinazolin-3-yl]-methanesulfonamide;
N-[7-Difluoromethyl-6-(2-isopropyl-2H-pyrazol-3-yl)-2,4-dioxo-1,4-dihydro-2H-quinazolin-3-yl]-methanesulfonamide;
N-[7-Ethyl-6-(2-methyl-2H-pyrazol-3-yl)-2,4-dioxo-1,4-dihydro-2H-quinazolin-3-yl]-methanesulfonamide;
N-[7-Ethyl-6-(2-ethyl-2H-pyrazol-3-yl)-2,4-dioxo-1,4-dihydro-2H-quinazolin-3-yl]-methanesulfonamide;
N-[7-Fluoromethyl-6-(2-methyl-2H-pyrazol-3-yl)-2,4-dioxo-1,4-dihydro-2H-quinazolin-3-yl]-methanesulfonamide;
N-[7-(1-fluoro-ethyl)-6-(2-methyl-2H-pyrazol-3-yl)-2,4-dioxo-1,4-dihydro-2H-quinazolin-3-yl]-methanesulfonamide;
N-[7-(1,1-difluoro-ethyl)-6-(2-methyl-2H-pyrazol-3-yl)-2,4-dioxo-1,4-dihydro-2H-quinazolin-3-yl]-methanesulfonamide;
N-[7-(1,1-difluoro-ethyl)-6-(2-isopropyl-2H-pyrazol-3-yl)-2,4-dioxo-1,4-dihydro-2H-quinazolin-3-yl]-methanesulfonamide;
N-[7-(1-fluoro-ethyl)-6-(2-isopropyl-2H-pyrazol-3-yl)-2,4-dioxo-1,4-dihydro-2H-quinazolin-3-yl]-methanesulfonamide; or
N-[6-(2-Methyl-2H-pyrazol-3-yl)-2,4-dioxo-7-trifluoromethyl-1,4-dihydro-2H-quinazolin-3-yl]-methanesulfonamide.

5. A compound of formula (I), their pharmaceutically acceptable salts and prodrugs thereof, according to claim 1 selected from the group consisting of:
N-[6-(1-Hydroxy-ethyl)-2,4-dioxo-7-trifluoromethyl-1,4-dihydro-2H-quinazolin-3-yl]-methanesulfonamide;
N-[6-(1-Methoxy-ethyl)-2,4-dioxo-7-trifluoromethyl-1,4-dihydro-2H-quinazolin-3-yl]-methanesulfonamide;
N-[6-(1-Hydroxy-propyl)-2,4-dioxo-7-trifluoromethyl-1,4-dihydro-2H-quinazolin-3-yl]-methanesulfonamide;
N-[6-(1-Isopropoxy-ethyl)-2,4-dioxo-7-trifluoromethyl-1,4-dihydro-2H-quinazolin-3-yl]-methanesulfonamide;
N-[6-(1-Ethoxy-ethyl)-2,4-dioxo-7-trifluoromethyl-1,4-dihydro-2H-quinazolin-3-yl]-methanesulfonamide;
N-[2,4-Dioxo-6-(1-propoxy-propyl)-7-trifluoromethyl-1,4-dihydro-2H-quinazolin-3-yl]-methanesulfonamide;
N-[6-(1-isopropoxy-propyl)-2,4-dioxo-7-trifluoromethyl-1,4-dihydro-2H-quinazolin-3-yl]-methanesulfonamide;
N-[6-(1-Cyclopentyloxy-ethyl)-2,4-dioxo-7-trifluoromethyl-1,4-dihydro-2H-quinazolin-3-yl]-methanesulfonamide
N-[6-(1-Hydroxy-butyl)-2,4-dioxo-7-trifluoromethyl-1,4-dihydro-2H-quinazolin-3-yl]-methanesulfonamide;
N-[6-(1-Hydroxy-3-methoxy-propyl)-2,4-dioxo-7-trifluoromethyl-1,4-dihydro-2H-quinazolin-3-yl]-methanesulfonamide;
N-[2,4-Dioxo-6-(tetrahydro-furan-2-yl)-7-trifluoromethyl-1,4-dihydro-2H-quinazolin-3-yl]-methanesulfonamide;
N-[2,4-Dioxo-6-(tetrahydro-furan-3-yl)-7-trifluoromethyl-1,4-dihydro-2H-quinazolin-3-yl]-methanesulfonamide;
N-{7-Isopropyl-6-[2-(2-methoxy-ethyl)-2H-pyrazol-3-yl]-2,4-dioxo-1,4-dihydro-2H-quinazolin-3-yl}-methanesulfonamide;
N-[6-(2-Isopropyl-2H-pyrazol-3-yl)-2,4-dioxo-7-trifluoromethyl-1,4-dihydro-2H-quinazolin-3-yl]-methanesulfonamide;
N-[7-Fluoromethyl-6-(2-isopropyl-2H-pyrazol-3-yl)-2,4-dioxo-1,4-dihydro-2H-quinazolin-3-yl]-methanesulfonamide;
N-{6-[2-(2-Methoxy-ethyl)-2H-pyrazol-3-yl]-2,4-dioxo-7-trifluoromethyl-1,4-dihydro-2H-quinazolin-3-yl}-methanesulfonamide;
N-[6-(2-Hydroxy-2H-pyrazol-3-yl)-2,4-dioxo-7-trifluoromethyl-1,4-dihydro-2H-quinazolin-3-yl]-methanesulfonamide;
N-[7-Ethyl-6-(2-isopropyl-2H-pyrazol-3-yl)-2,4-dioxo-1,4-dihydro-2H-quinazolin-3-yl]-methanesulfonamide;
N-[7-Isopropyl-6-(2-methyl-2H-pyrazol-3-yl)-2,4-dioxo-1,4-dihydro-2H-quinazolin-3-yl]-methanesulfonamide;
N-[7-Isopropyl-6-(2-isopropyl-2H-pyrazol-3-yl)-2,4-dioxo-1,4-dihydro-2H-quinazolin-3-yl]-methanesulfonamide;
N-[7-Difluoromethyl-6-(2-methyl-2H-pyrazol-3-yl)-2,4-dioxo-1,4-dihydro-2H-quinazolin-3-yl]-methanesulfonamide;
N-[7-Difluoromethyl-6-(2-isopropyl-2H-pyrazol-3-yl)-2,4-dioxo-1,4-dihydro-2H-quinazolin-3-yl]-methanesulfonamide;
N-[7-Ethyl-6-(2-methyl-2H-pyrazol-3-yl)-2,4-dioxo-1,4-dihydro-2H-quinazolin-3-yl]-methanesulfonamide;
N-[7-Ethyl-6-(2-ethyl-2H-pyrazol-3-yl)-2,4-dioxo-1,4-dihydro-2H-quinazolin-3-yl]-methanesulfonamide;
N-[7-(1,1-difluoro-ethyl)-6-(2-methyl-2H-pyrazol-3-yl)-2,4-dioxo-1,4-dihydro-2H-quinazolin-3-yl]-methanesulfonamide;
N-[7-(1,1-difluoro-ethyl)-6-(2-isopropyl-2H-pyrazol-3-yl)-2,4-dioxo-1,4-dihydro-2H-quinazolin-3-yl]-methanesulfonamide
N-[7-(1-fluoro-ethyl)-6-(2-isopropyl-2H-pyrazol-3-yl)-2,4-dioxo-1,4-dihydro-2H-quinazolin-3-yl]-methanesulfonamide; and
N-[6-(2-Methyl-2H-pyrazol-3-yl)-2,4-dioxo-7-trifluoromethyl-1,4-dihydro-2H-quinazolin-3-yl]-methanesulfonamide.

6. A compound of formula (I), their pharmaceutically acceptable salts and prodrugs thereof, according to claim 1, selected from the group consisting of:
N-[6-(1-Hydroxy-ethyl)-2,4-dioxo-7-trifluoromethyl-1,4-dihydro-2H-quinazolin-3-yl]-methanesulfonamide;
N-[6-(1-Methoxy-ethyl)-2,4-dioxo-7-trifluoromethyl-1,4-dihydro-2H-quinazolin-3-yl]-methanesulfonamide;
N-[6-(1-Hydroxy-propyl)-2,4-dioxo-7-trifluoromethyl-1,4-dihydro-2H-quinazolin-3-yl]-methanesulfonamide;
N-[6-(1-Isopropoxy-ethyl)-2,4-dioxo-7-trifluoromethyl-1,4-dihydro-2H-quinazolin-3-yl]-methanesulfonamide;
N-[6-(1-Ethoxy-ethyl)-2,4-dioxo-7-trifluoromethyl-1,4-dihydro-2H-quinazolin-3-yl]-methanesulfonamide;
N-[6-(1-isopropoxy-propyl)-2,4-dioxo-7-trifluoromethyl-1,4-dihydro-2H-quinazolin-3-yl]-methanesulfonamide;
N-[6-(1-Hydroxy-butyl)-2,4-dioxo-7-trifluoromethyl-1,4-dihydro-2H-quinazolin-3-yl]-methanesulfonamide;
N-[2,4-Dioxo-6-(tetrahydro-furan-2-yl)-7-trifluoromethyl-1,4-dihydro-2H-quinazolin-3-yl]-methanesulfonamide;
N-[2,4-Dioxo-6-(tetrahydro-furan-3-yl)-7-trifluoromethyl-1,4-dihydro-2H-quinazolin-3-yl]-methanesulfonamide;
N-{7-Isopropyl-6-[2-(2-methoxy-ethyl)-2H-pyrazol-3-yl]-2,4-dioxo-1,4-dihydro-2H-quinazolin-3-yl}-methanesulfonamide;
N-[6-(2-Isopropyl-2H-pyrazol-3-yl)-2,4-dioxo-7-trifluoromethyl-1,4-dihydro-2H-quinazolin-3-yl]-methanesulfonamide;
N-[7-Fluoromethyl-6-(2-isopropyl-2H-pyrazol-3-yl)-2,4-dioxo-1,4-dihydro-2H-quinazolin-3-yl]-methanesulfonamide;
N-[7-Ethyl-6-(2-isopropyl-2H-pyrazol-3-yl)-2,4-dioxo-1,4-dihydro-2H-quinazolin-3-yl]-methanesulfonamide;
N-[7-Isopropyl-6-(2-methyl-2H-pyrazol-3-yl)-2,4-dioxo-1,4-dihydro-2H-quinazolin-3-yl]-methanesulfonamide;
N-[7-Isopropyl-6-(2-isopropyl-2H-pyrazol-3-yl)-2,4-dioxo-1,4-dihydro-2H-quinazolin-3-yl]-methanesulfonamide;
N-[7-Difluoromethyl-6-(2-methyl-2H-pyrazol-3-yl)-2,4-dioxo-1,4-dihydro-2H-quinazolin-3-yl]-methanesulfonamide;
N-[7-Difluoromethyl-6-(2-isopropyl-2H-pyrazol-3-yl)-2,4-dioxo-1,4-dihydro-2H-quinazolin-3-yl]-methanesulfonamide;
N-[7-Ethyl-6-(2-methyl-2H-pyrazol-3-yl)-2,4-dioxo-1,4-dihydro-2H-quinazolin-3-yl]-methanesulfonamide;
N-[7-Ethyl-6-(2-ethyl-2H-pyrazol-3-yl)-2,4-dioxo-1,4-dihydro-2H-quinazolin-3-yl]-methanesulfonamide;
N-[7-(1,1-difluoro-ethyl)-6-(2-methyl-2H-pyrazol-3-yl)-2,4-dioxo-1,4-dihydro-2H-quinazolin-3-yl]-methanesulfonamide;
N-[7-(1-fluoro-ethyl)-6-(2-isopropyl-2H-pyrazol-3-yl)-2,4-dioxo-1,4-dihydro-2H-quinazolin-3-yl]-methanesulfonamide; and
N-[6-(2-Methyl-2H-pyrazol-3-yl)-2,4-dioxo-7-trifluoromethyl-1,4-dihydro-2H-quinazolin-3-yl]-methanesulfonamide.

7. A compound of formula (I), their pharmaceutically acceptable salts and prodrugs thereof, according to claim 1, selected from the group consisting of:
N-[6-(1-Methoxy-ethyl)-2,4-dioxo-7-trifluoromethyl-1,4-dihydro-2H-quinazolin-3-yl]-methanesulfonamide;
N-[6-(1-Ethoxy-ethyl)-2,4-dioxo-7-trifluoromethyl-1,4-dihydro-2H-quinazolin-3-yl]-methanesulfonamide;
N-[7-Isopropyl-6-(2-methyl-2H-pyrazol-3-yl)-2,4-dioxo-1,4-dihydro-2H-quinazolin-3-yl]-methanesulfonamide;
N-[2,4-Dioxo-6-(tetrahydro-furan-2-yl)-7-trifluoromethyl-1,4-dihydro-2H-quinazolin-3-yl]-methanesulfonamide;
N-[7-Difluoromethyl-6-(2-methyl-2H-pyrazol-3-yl)-2,4-dioxo-1,4-dihydro-2H-quinazolin-3-yl]-methanesulfonamide;
N-[7-Ethyl-6-(2-methyl-2H-pyrazol-3-yl)-2,4-dioxo-1,4-dihydro-2H-quinazolin-3-yl]-methanesulfonamide;

8. A compound of formula (I), their pharmaceutically acceptable salts and prodrugs thereof, according to claim 1, wherein the compound of formula (I) is N-[7-Isopropyl-6-(2-methyl-2H-pyrazol-3-yl)-2,4-dioxo-1,4-dihydro-2H-quinazolin-3-yl]-methanesulfonamide, or a pharmaceutically acceptable salt thereof.

9. A compound of formula (I), their pharmaceutically acceptable salts and prodrugs thereof, according to claim 1, wherein the compound of formula (I) is N-[6-(1-Methoxy-ethyl)-2,4-dioxo-7-trifluoromethyl-1,4-dihydro-2H-quinazolin-3-yl]-methanesulfonamide

10. A compound of formula (I), their pharmaceutically acceptable salts and prodrugs thereof, according to claim 1, wherein the compound of formula (I) is N-[6-(1-Ethoxy-ethyl)-2,4-dioxo-7-trifluoromethyl-1,4-dihydro-2H-quinazolin-3-yl]-methanesulfonamide.

11. A compound of formula (I), their pharmaceutically acceptable salts and prodrugs thereof, according to claim 1, wherein the compound of formula (I) is N-[7-Ethyl-6-(2-methyl-2H-pyrazol-3-yl)-2,4-dioxo-1,4-dihydro-2H-quinazolin-3-yl]-methanesulfonamide

12. (canceled)

13. A method for the treatment, prevention or delay of progression of neuronal ceroid lipofuscinosis in a subject in need of such treatment, which comprises administering to said subject a therapeutically effective amount of a compound of formula (I), their pharmaceutically acceptable salts and prodrugs thereof, as defined in claim 1.

14. A pharmaceutical composition comprising a compound of formula (I), their pharmaceutically acceptable salts and prodrugs thereof, as defined in claim 1; for the treatment, prevention or delay of progression of neuronal ceroid lipofuscinosis.

15. A compound of formula (I), their pharmaceutically acceptable salts and prodrugs thereof, according to claim 1 for the treatment, prevention or delay of progression of neuronal ceroid lipofuscinosis selected from:
Infantile neuronal ceroid lipofuscinoses (INCL);
Late infantile neuronal ceroid lipofuscinoses (LINCL);
Juvenile neuronal ceroid lipofuscinoses (JNCL);
Adult neuronal ceroid lipofuscinoses (ANCL);
Finnish Late Infantile neuronal ceroid lipofuscinoses (fLINCL);
Portuguese Late Infantile neuronal ceroid lipofuscinoses (pLINCL);
Turkish Late Infantile neuronal ceroid lipofuscinoses; and neuronal ceroid lipofuscinoses (tLINCL); and Progressive Epilepsy with Mental Retardation neuronal ceroid lipofuscinoses (EPMR).
